(19) United States
(12) Patent Application Publication
(10) Pub. No.: US 2010/0305474 A1
(43) Pub. Date: Dec. 2, 2010

(54) APPARATUS FOR DISPENSING A WIRE GUIDE

(75) Inventors: Bruce J. DeMars, Bloomington, IN (US); Gregory A. Frankland, Unionville, IN (US); Alan A. Petrucci, Western Springs, IL (US)

Correspondence Address: BRINKS HOFER GILSON & LIONE/INDY/COOK;BRINKS HOFER GILSON & LIONE, CAPITAL CENTER, SUITE 1100, 201 NORTH ILLINOIS STREET, INDIANAPOLIS, IN, 46204-4220, US

(73) Assignee: Cook Critical Care Incorporated, Bloomington, IN (US)

(21) Appl. No.: 12/472,778

(22) Filed: May 27, 2009

(51) Int. Cl. A61M 25/00; B65D 85/04; B65H 49/06

(52) U.S. Cl. 600/585; 242/588.3; 242/588.2

(57) ABSTRACT

An apparatus for dispensing a wire guide into a body vessel of a patient includes a first apparatus half and a second apparatus half. Each of the apparatus halves has an interior surface, wherein each of the halves is structured and aligned for joinder with the other half along the respective interior surfaces. Upon joinder of the apparatus halves, the resulting apparatus defines a generally spiral-shaped interior wire guide pathway, a wire guide opening generally tangential to the interior pathway, a guideway for receiving an end of the wire guide from the opening, a first port communicating with the pathway, and a second port communicating with the first port.

### BACKGROUND

1. Technical Field

This invention relates generally to an apparatus for use in the medical arts. More particularly, the invention relates to an apparatus for use in holding a wire guide, and from which the wire guide can be controllably dispensed into a body vessel for carrying out a medical procedure.

2. Background Information

In recent years, the use of minimally invasive techniques for accessing previously hard-to-reach areas within the body of a patient has exploded. In many instances, such access is gained by utilizing percutaneous entry techniques, such as the well-known Seldinger technique. In the Seldinger technique, a needle is initially inserted into a body vessel. A wire guide is then inserted into the vessel through a bore in the needle, and the needle is withdrawn. The wire guide is threaded through the body vessel to, or near, a target site for carrying out a medical procedure. A catheter, sheath, or other medical device may then be passed over the wire guide and directed through the vessel until the device accesses the target site. Typically, the wire guide is thereafter withdrawn from the vessel, leaving the medical device in place for use in carrying out the intended procedure. Among the many procedures that may be carried out in this manner are the delivery of another medical device, such as a stent, to the target area, and the delivery of a fluid to the target area, e.g., for diagnostic or treatment purposes.

The wire guide can be inserted into the body vessel by any of a number of known techniques. On some occasions, such as when a remote region within the body of the patient is to be accessed, an elongated wire guide may initially extend several feet outside of the body of the patient as the distal end of the wire guide is initially inserted into the passageway by the physician. In this event, a physician's assistant may be required to maintain control of the extended portion of this wire guide, and to safeguard the extended portion from contamination prior to insertion into the body passageway.

On other occasions, in order to avoid the necessity of having an elongated end of a wire guide extending outside the patient as described, a wire guide holder, or dispenser, may be utilized. The wire guide is typically wound or otherwise stored in the dispenser prior to use, and dispensed therefrom into the vessel. Such wire guide holders are often difficult to maneuver, are not ergonomically efficient, render the wire guide difficult to lubricate, and do not provide adequate means for easily and accurately dispensing the wire guide therefrom. Furthermore, some wire guide holders are difficult to manufacture and load with the wire guide.

It would be desirable to provide an apparatus that is capable of controllably dispensing a wire guide into the body vessel, and that overcomes the problems encountered with prior art devices.

### BRIEF SUMMARY

The present invention addresses the problems of the prior art. In one form thereof, the invention comprises an apparatus for dispensing a wire guide into a body vessel of a patient. The apparatus includes a first apparatus half and a second apparatus half. Each of the apparatus halves has an interior surface, wherein each of the halves is structured and aligned for joinder with the other half along the respective interior surfaces. Upon joinder of the apparatus halves, the resulting apparatus defines a generally spiral-shaped interior wire guide pathway, a wire guide opening generally tangential to the interior pathway, a guideway for receiving an end of the wire guide from the opening, a first port communicating with the pathway, and a second port communicating with the pathway and the first port.

In another form thereof, the invention comprises a wire guide dispensing apparatus. The dispensing apparatus comprises a dispenser body comprising a first half and a second half. The respective first and second halves are structured and arranged such that upon joinder thereof an interior wire guide pathway is defined thereby. The dispenser body includes a wire guide opening configured for passage of the wire guide from the pathway. The dispenser body further includes a first port communicating with the pathway for receiving a flushing fluid for the wire guide, and a second port communicating with the first port for drainage of the flushing fluid from the pathway.

### BRIEF DESCRIPTION OF THE DRAWINGS

FIG. 1 is perspective view of an apparatus for dispensing a wire guide according to an embodiment of the present invention;

FIG. 2 is side view of the dispensing apparatus of FIG. 1;

FIG. 3 is a top view of the dispensing apparatus of FIG. 1, taken from the orientation of FIG. 2;

FIG. 4 is an internal view of one side of the dispensing apparatus of FIG. 1, illustrating the generally spiral-shaped pathway for the wire guide;

FIG. 5 is a sectional view taken from line 5-5 of FIG. 1; and

FIG. 6 is a perspective side view of the dispensing apparatus of FIG. 1, illustrating a manner of use of the apparatus by an operator, and further illustrating a wire guide straightener and an end of the wire guide being dispensed from the apparatus.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

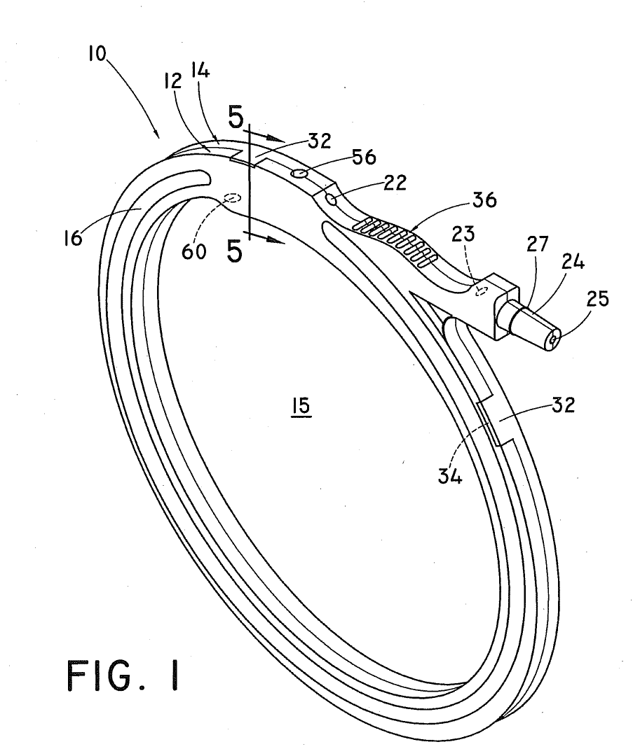

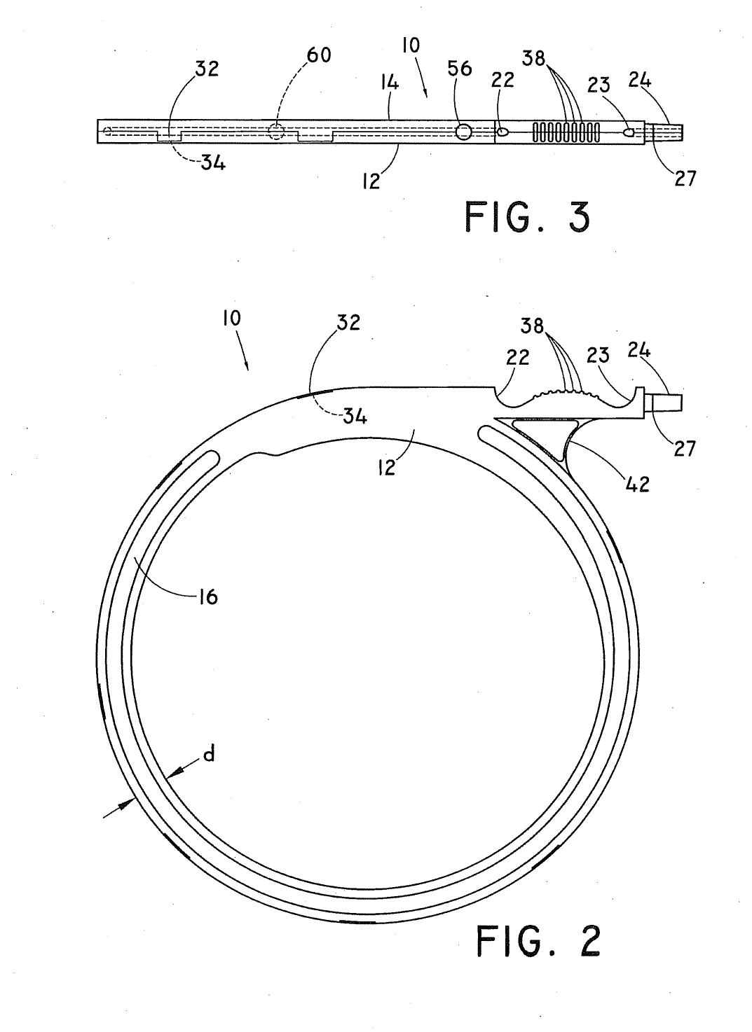

FIG. 1 illustrates a perspective view of an apparatus 10 for dispensing a wire guide. In the embodiment shown, apparatus 10 comprises two apparatus halves 12, 14, that may be snapped or otherwise fitted together. FIG. 2 illustrates a side view of apparatus half 12. Each of halves 12, 14 includes an optional circumferential groove 16 extending along a portion of the outer circumference of the apparatus half. Optional groove 16 makes the apparatus easier to grip, facilitates molding, and provides strength to the assembled apparatus.

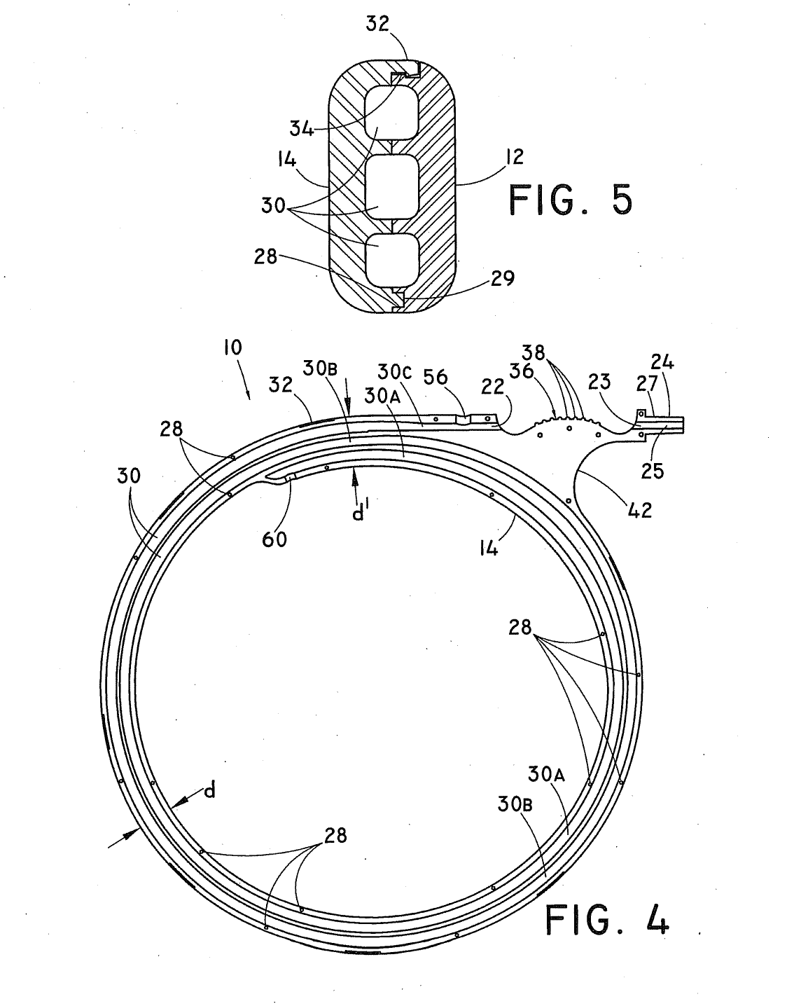

Other features of apparatus 10 are more prominent in FIGS. 3-5. FIG. 3 illustrates a top view of apparatus 10, taken from the orientation shown in FIG. 2. FIG. 4 illustrates an internal view of apparatus half 14, prior to joinder with apparatus half 12. FIG. 5 illustrates a sectional view taken from line 5-5 of FIG. 1.

As shown in FIGS. 4 and 5, a generally spiral-shaped pathway 30 winds along the interior of apparatus 10. Each of apparatus halves 12, 14 includes a portion of pathway 30, such that when apparatus 10 is assembled as shown in FIG. 1, the spiral-shaped pathway is defined thereby. When apparatus 10 is fully assembled, a wire guide (not shown) extends along the spiral-shaped pathway, and a distal end of the wire guide extends out of the apparatus at opening 22 formed at the end of the spiral-shaped pathway, as described in greater detail herein. Preferably, opening 22 is oriented generally tangential to the interior pathway.

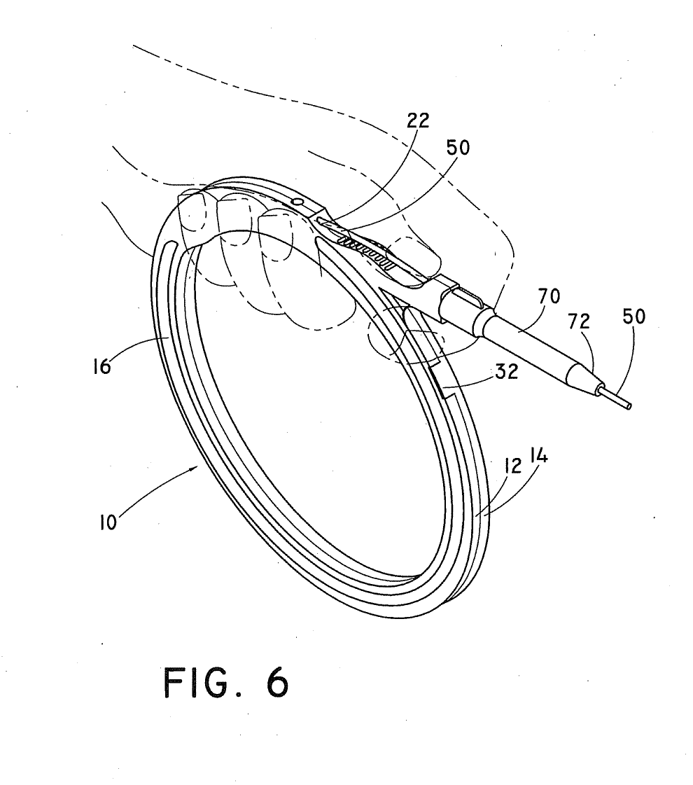

In the preferred embodiment shown, apparatus halves 12,14 are generally ring-shaped, and include an opening 15 (FIG. 1) at a center portion of the apparatus half. This configuration facilitates grasping of the apparatus 10 by the fingers of the user, as shown in FIG. 6. It also provides for an efficient size and shape of the apparatus, as there is little wasted space in the apparatus, and the configuration is conducive to forming the spiral-shaped pathway as described. Although this shape is preferred, those skilled in the art will appreciate that other configurations having an opening suitable for grasping by the user at a center portion thereof, such as an oval, elliptical, square, etc., may be substituted.

In the embodiment shown, apparatus half 14 includes a plurality of posts 28 situated along the interior surface of the apparatus half. The interior of apparatus half 12 includes a plurality of apertures (not shown) that correspond to posts 28. When apparatus 10 is fully assembled and halves 12, 14 are snapped together, each of posts 28 is received in a separate aperture.

In addition to the snap-fit of posts 28 into the apertures as described, or as an alternative-to this feature, apparatus 10 is provided with another means for engaging halves 12, 14. As best shown in FIG. 1, one of the apparatus halves, in this case apparatus half 14 is provided with a series of clips 32 along its outer circumference. The other apparatus half, in this case apparatus half 12, is provided with a series of grooves 34 along its outer surface. Clips 32 and grooves 34 are sized and spaced along such circumference such that they meet to provide a snap-fit for apparatus 10 upon assembly.

Those skilled in the art will appreciate that the use and arrangement of the posts and apertures, and/or the clips and grooves, respectively, as described and shown herein for forming a snap-fit between respective apparatus halves is not intended to be exclusive, and represents only one example of numerous possible ways to accomplish said fitting and assembly. Other methods well-known to those skilled in the art for accomplishing said fitting and assembly may be substituted, and are considered within the scope of the invention.

Apparatus 10 generally has a diameter d along the major portion of its circumference. At portions of apparatus 10 having diameter d, the spiral-shaped pathway 30 includes two turns 30A, 30B (FIG. 4), respectively for receipt of the wire guide. At a dispensing portion of the embodiment of apparatus 10 shown in FIG. 4, spiral-shaped pathway 30 has a larger diameter d′. This portion of apparatus 10 includes three wire guide turns 30A, 30B, 30C, respectively. Wire guide turn 30C terminates in wire guide opening 22.

Apparatus 10 further includes a guideway 36 generally adjacent opening 22. In the non-limiting embodiment shown, guideway 36 is provided with a gentle curvature, and a series of optional projections, such as ribs 38, spaced therealong. The presence of ribs 38 and the curvature of guideway 36 provide ergonomic benefits, as they facilitate receipt of the operator's thumb (FIG. 6). This arrangement provides enhanced user comfort and control when dispensing and/or retracting the wire guide between wire guide openings 22 and 23. The presence of the ribs 38 also inhibits slippage of the wire.

Each of halves 12, 14 is preferably shaped such that a finger grip 42 is defined upon engagement of the halves. Finger grip 42 will be further discussed herein. Additionally, apparatus 10 is preferably provided with a spout 24. Spout 24 is positioned generally adjacent guideway 36, and situated at the opposite axial end of guideway 36 from opening 22. Spout 24 has a passageway 25 extending therethrough for passage of the wire guide.

A feature of apparatus 10 is the presence of an injection, or flushing, port 56 at a top surface of apparatus 10. An exit port 60 is provided at an opposing side of apparatus 10. These ports are best shown in FIG. 4. Injection port 56 is preferably configured to accept a standard syringe (not shown), so that a flushing solution may be injected into the port from the syringe. The flushing solution allows for lubrication of the wire guide, whether coated or uncoated. In prior art wire guide holders, the wire guide is typically dispensed from the holder and dipped in water basin prior to insertion into the patient. Providing a port sized and oriented for receiving the flushing solution provides a more convenient, and sterile, manner of lubricating the wire guide. Exit port 60 is located downstream of the injection port for removal or flushing of excess medium. In the preferred embodiment, exit port 60 is located at an end of larger diameter portion d′ opposite wire guide opening 22.

FIG. 6 illustrates the use of dispensing apparatus 10 by an operator (operator hand shown in broken lines) to dispense a wire guide 50. In this figure, an optional wire guide straightener 70 having a tapered distal tip portion 72 has been positioned at the end of spout 24. The use of a wire guide straightener straightens out the J-tip of a typical wire guide, thereby facilitating entry of the wire guide into, e.g., the bore of a needle. Wire guide straighteners are generally cylindrical elongated structures having a passageway therethrough for the wire guide, and having a tapered distal tip portion. Wire guide straighteners are well known in the art, and straightener 70 may be of any known type, as long as it is dimensioned to fit onto the end of the spout. Preferably, spout 24 is provided with an optional structure, such as snap ring 27, that is sized to form a snap fit with a corresponding snap ring (not shown) in the barrel of wire guide straightener 70. The presence of the complementary structure (such as the snap rings) on the spout and wire guide straightener minimizes the possibility of unintended detachment of the straightener from the wire guide holder.

During use of apparatus 10, an end of the wire guide 50 extends through opening 22, and further extends along the ribs of guideway 36 to opening 23. Wire guide 50 thereafter extends through passageway 25 of spout 24. As described, the end of wire guide 50 then passes through wire guide straightener 70, and exits straightener 70 as shown in FIG. 6.

As illustrated in FIG. 6, the user simply grasps apparatus 10 as shown. The index finger is positioned along finger grip 42, and the remaining fingers generally encircle a portion of apparatus 10. Preferably, finger grip 42 is configured in the nature of a trigger, such that it readily accepts the user's index finger in well-known manner. The wire guide is threaded between the thumb of the user and the ribbed guideway 36. As a result of the inventive configuration, the user may easily advance the wire guide, by forward motion of the thumb, in controlled fashion along guideway 36 and beyond the distal tip 72 of straightener 70. Alternatively, by reverse thumb motion, the user may retract the wire guide back into apparatus 10.

Those skilled in the art will appreciate that the wire guide dispenser of the present invention may be constructed by molding or otherwise forming the apparatus halves 12, 14, and thereafter simply attaching the individual halves together, such as by the snap fit previously described. Although it is believed that the individual halves 12, 14 may be most conveniently formed by molding the individual pieces in conventional manner, other well-known methods for forming devices may be substituted.

Preferably, the apparatus 10 will be formed from a rigid, or semi-rigid polymer. Polypropylene, polyamide, and polyethylene are non-limiting examples of suitable polymers. Also, filled polymers, such as talc-filled polypropylene, are suitable due to their stiffness, hardness, and dimensional stability. Those skilled in the art will appreciate that any material capable of housing a wire guide and capable of being formed to facilitate gripping by a user may be substituted.

It is therefore intended that the foregoing detailed description be regarded as illustrative rather than limiting, and that it be understood that it is the following claims, including all equivalents, that are intended to define the spirit and scope of this invention.

1. An apparatus for dispensing a wire guide into a body vessel of a patient, comprising:
a first apparatus half and a second apparatus half, each of said first and second apparatus halves having an interior surface, each of said halves structured and aligned for joinder with the other of said halves along said respective interior surfaces such that a generally spiral-shaped interior wire guide pathway, a wire guide opening generally tangential to said interior pathway, a guideway for receiving an end of said wire guide from said opening, a first port communicating with said pathway, and a second port communicating with said first port, are defined thereby.

2. The apparatus of claim 1, each of said apparatus halves being generally ring-shaped and having an opening at a center portion of said apparatus half, said center portion openings configured and aligned such that said apparatus is graspable by fingers of a user extending into said opening upon joinder of said apparatus halves.

3. The apparatus of claim 2, wherein each of said apparatus halves is structured and aligned to define a finger grip for receiving an index finger of a user upon joinder of said apparatus halves.

4. The apparatus of claim 1, wherein each of said apparatus halves is structured and aligned to define a spout, said spout positioned at an opposite end of said guideway from said generally tangential wire guide opening.

5. The apparatus of claim 1, wherein said apparatus halves are provided with complementary structure for effecting joinder of said apparatus halves.

6. The apparatus of claim 5, wherein said complementary structure comprises a series of posts on said interior surface of one of said apparatus halves, and a series of apertures on the interior surface of the other of said apparatus halves for receiving said posts.

7. The apparatus of claim 5, wherein said complementary structure comprises a series of clips on one of said apparatus halves, and a series of complementary grooves on the other of said halves for receiving said clips.

8. The apparatus of claim 5, wherein said complementary structure comprises a series of posts on said interior surface of one of said apparatus halves, and a series of apertures on the other of said apparatus halves for receiving said posts; and a series of clips on one of said apparatus halves, and a series of complementary grooves on the other of said halves for receiving said clips.

9. The apparatus of claim 1, wherein said first port is disposed generally adjacent said wire guide opening, and said second port is disposed generally opposite said first port.

10. The apparatus of claim 1, wherein said guideway includes a plurality of ribs spaced therealong.

11. The apparatus of claim 1, wherein each of said apparatus halves has an exterior surface, and further includes a circumferential groove extending along at least a portion of said exterior surface.

12. A wire guide dispensing apparatus, comprising:
a dispenser body comprising a first half and a second half, said first and second halves structured and arranged such that upon joinder thereof an interior wire guide pathway is defined thereby, said dispenser body including a wire guide opening configured for passage of said wire guide from said pathway, said dispenser body further including a first port communicating with said pathway for receiving a flushing fluid for said wire guide, and a second port communicating with said first port for drainage of said flushing fluid from said pathway.

13. The wire guide dispensing apparatus of claim 12, wherein said dispenser body further includes a guideway generally adjacent said wire guide opening for receiving said wire guide, and a spout positioned at an opposite end of said guideway from said wire guide opening.

14. The wire guide dispensing apparatus of claim 13, wherein said guideway includes a plurality of ribs spaced therealong.

15. The wire guide dispensing apparatus of claim 13, wherein said spout includes a snap ring along an outer surface thereof, further comprising a wire guide straightener engaged with said spout at said snap ring.

16. The wire guide dispensing apparatus of claim 12, wherein said dispenser body halves are provided with complementary structure for effecting joinder thereof.

17. The wire guide dispensing apparatus of claim 16, wherein said complementary structure comprises a series of posts on said interior surface of one of said dispenser body halves, and a series of apertures on the interior surface of the other of said dispenser body halves for receiving said posts.

18. The wire guide dispensing apparatus of claim 17, wherein said complementary structure further comprises a series of clips on one of said halves, and a series of complementary grooves on the other of said halves for receiving said clips.

19. The wire guide dispensing apparatus of claim 13, each of said halves being generally ring-shaped and having an opening at a center portion thereof, said center portion openings configured and aligned such that said apparatus is graspable by fingers of a user extending into said opening.

20. The wire guide dispensing apparatus of claim 19, wherein each of said halves is structured and aligned to define a finger grip for receiving an index finger of a user upon joinder thereof.
